(19) United States
(12) Patent Application Publication
(10) Pub. No.: US 2023/0270601 A1
(43) Pub. Date: Aug. 31, 2023

(54) ARTICLE WITH IMPROVED ELASTOMERIC WAISTBAND

(71) Applicant: Kimberly-Clark Worldwide, Inc., Neenah, WI (US)

(72) Inventors: EunHye Cho, London (GB); Dmitry Vnukov, Stupino (RU)

(21) Appl. No.: 18/016,522

(22) PCT Filed: Jul. 21, 2020
(86) PCT No.: PCT/US2020/042919

§ 371 Date: Jan. 17, 2023

(51) Int. Cl. A61F 13/49; A61F 13/496

(52) U.S. Cl. CPC A61F 13/49012; A61F 13/496; A61F 2013/15292

(57) ABSTRACT

Absorbent garments and methods of manufacture are disclosed. An absorbent garment may comprise front, back, and crotch regions, an absorbent assembly, a front panel and a back panel, the front panel and back panel each including an inner layer and an outer layer, with a stranded elastic area disposed between the inner layers and the outer layers, a front panel stretch band material disposed between the outer layer and a portion of the inner layer, with the stranded elastic area adjacent and discrete from and disposed closer to the crotch region than the stretch band material. The article further comprising a stretch band material adhesive coupling the stretch band material to the inner layer, and a longitudinal overlap between the stretch band material adhesive and the inner layer may be greater than about 40% of an overall longitudinal length of the stretch band material.

### TECHNICAL FIELD

The present disclosure is directed to absorbent articles and more specifically to absorbent articles with an improved waistband.

### BACKGROUND

Absorbent articles, such as diapers, incontinence garments, training pants, sanitary napkins, panty liners, and the like are well known in the art. These articles, which are often disposable, are capable of absorbing and retaining fluids and other bodily discharges. Some absorbent articles, such as pull-on type absorbent articles, include a central absorbent member and side panels extending from and interconnecting respective front and back regions of the absorbent member forming a waist assembly.

An example of a pull-on absorbent article is a diaper pant; diaper pants are typically worn by babies who are able to stand up, but who are not yet old enough to be toilet trained. Absorbent articles in the diaper pants format typically include a waist assembly (that includes side panels) formed of elastic strands placed between inner and outer layers of a nonwoven material. The elastic strands are typically kept in place by adhesive. These types of waist assemblies may include 20 to 30 high-tension elastic strands that embrace the waist area. The use of the elastic strands in the side panels of the diaper pants allows the side panels to be highly elastic and allows for a wide range of body shapes over which the diaper pant may be donned. The elastic side panels also provide a conforming fit for the wearer.

The high waist area of the waist assembly typically gets stretched the most in use in order to conform to the child’s belly/abdomen. If high-tension elastic strands are stretched excessively, they may exert a lot of pressure on the skin which could lead to red-marking on the skin caused by the diaper pant. The extended, high waist area of the diaper pant may have a wrinkled appearance in use; the wrinkled appearance may give an impression of being stuffy and uncomfortable. Further, the known construction of diaper pants does not provide an underwear-like appearance.

There remains a need for a diaper pant that provides a more skin-friendly contact in the high waist area. While providing a more skin-friendly contact, the waist assembly needs to remain very stretchable. There also remains a need for a diaper pant that has fewer wrinkles in the high waist area and, therefore, provides a smoother look around the waist. Further, there remains a need for a diaper pant that provides a more underwear-like appearance in use.

### SUMMARY

Significant improvements in appearance, comfort and fit of diaper pants may be achieved using a new construction for the waist assembly of the diaper pant. The waist assembly is typically formed by a front panel and a back panel of the diaper pant. The diaper pants of the present disclosure include a waist assembly having a lower waist area that includes stranded elastics and an upper waist area that includes a discrete, stretch banded area. The stranded elastic area includes an inner material and an outer material in between which the elastic strands are placed. The discrete, stretch banded area may be formed by placing a stretchable material (that has stretch in at least one direction; for example, in at least a longitudinal or a lateral direction) against the same outer material used to form the stranded elastic area and then folding the outer material over to cover the other side of the stretch banded material. In some embodiments, the stretchable material may overlap a portion of the inner material as well.

According to a first aspect, an absorbent garment may comprise a longitudinal axis and a lateral axis, a front region, a back region, a crotch region, the crotch region being disposed between the front region and the back region, and the crotch region including an absorbent assembly, a front panel in the front region, wherein the front panel includes an inner layer, an outer layer and a front longitudinal end, a back panel in the back region, wherein the back panel includes an inner layer, an outer layer and a back longitudinal end, a stranded elastic area disposed between the inner layer and the outer layer in the front panel and in the back panel, a stretch banded area comprising a stretch band material disposed between the outer layer and at least a portion of the inner layer in the front panel, the stretch band material disposed adjacent the longitudinal end of the front panel with the stranded elastic area adjacent and discrete from the stretch band material and disposed closer to the crotch region than the stretch band material, and a stretch band material adhesive coupling the stretch band material to the front panel inner layer, and wherein a longitudinal overlap between the stretch band material adhesive and the front panel inner layer may be greater than about 40% of an overall longitudinal length of the stretch band material.

According to a second aspect, an absorbent garment may comprise a longitudinal axis and a lateral axis, a front region, a back region, a crotch region, the crotch region being disposed between the front region and the back region, and the crotch region including an absorbent assembly, a front panel in the front region, wherein the front panel includes an inner layer, an outer layer and a front longitudinal end, the front panel inner layer comprising a material having a CD tensile strength that is greater than about 12N/50mm, a back panel in the back region, wherein the back panel includes an inner layer, an outer layer and a back longitudinal end, a stranded elastic area disposed between the inner layer and the outer layer in the front panel and in the back panel, a stretch banded area comprising a stretch band material disposed between the outer layer and at least a portion of the inner layer in the front panel, the stretch band material disposed adjacent the longitudinal end of the front panel with the stranded elastic area adjacent and discrete from the stretch band material and disposed closer to the crotch region than the stretch band material, an inner layer adhesive coupling the front panel inner layer to the front panel outer layer, and a stretch band material adhesive coupling the stretch band material to the front panel inner layer, and wherein a longitudinal overlap between the inner layer adhesive and the stretch band material adhesive may be greater than about 40% of an overall longitudinal length of the stretch band material.

### DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a top down plan view of an absorbent garment of the present disclosure;

FIG. 2 is a front view of the absorbent garment of FIG. 1, in an assembled condition;

FIG. 3 is a cross-sectional view of the absorbent garment of FIG. 1 taken along line “3-3” in FIG. 1; and

FIG. 4 is a close-up view of a portion of the cross-sectional view of FIG. 3.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

The term “about” refers herein to a range of values close in proximity to the stated value. More specifically, the term “about” may modify the stated value by up to 5% of the stated value, within the same unit space. As one example, the phrase “about 10 gsm” may be read to include a range of between 9.5 gsm and 10.5 gsm. As another example, the phrase “about 40%” may be read in include a range of between of 38% and 42%.

The term “absorbent garment” refers herein to a garment that can be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent garments, including, but not limited to, infant diaper pants, toilet-training pants, youth pants, swim pants, feminine hygiene garments and adult incontinence garments.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements.

The term “carded web” refers herein to a web containing natural or synthetic staple fibers typically having fiber lengths less than about 100 mm. Bales of staple fibers can undergo an opening process to separate the fibers that are then sent to a carding process that separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers can be subject to adhesive processes to bind the fibers together such as by the use of powder adhesives. The carded web can be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films that constitute liquid transfer films, as well as films that do not transfer fluids, such as, but not limited to, barrier films, filled films, breathable films, and oriented films.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams that attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers that can be continuous or discontinuous, are generally smaller than about 0.6 denier, and can be tacky and self-bonding when deposited onto a collecting surface.

The term “nonwoven” refers herein to materials and webs of material that are formed without the aid of a textile weaving or knitting process. The materials and webs of materials can have a structure of individual fibers, filaments, or threads (collectively referred to as “fibers”) that can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials that are compliant and that will readily conform to the general shape and contours of the wearer’s body.

The term “spunbond” refers herein to small diameter fibers that are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an aspect, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an aspect, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a polymeric material that becomes pliable or moldable above a specific temperature and returns to a solid state upon cooling.

Generally, an absorbent garment having an improved structure is disclosed herein. The improved structure provides a waistband area that is part of an overall waist assembly. The absorbent garments of the present disclosure provide the wearers of the garments with a more comfortable fit because there is less retraction of the elastics in the overall waist assembly. The absorbent garments of the present disclosure have a waist assembly structure that includes discrete elastic zones. Conventional absorbent garments have waist assemblies that include either elastic strands by themselves or elastic strands combined with other elastics, but with the elastic strands used throughout the waist assembly. With the absorbent garments of the present disclosure, the waist assembly includes a stretch banded area that is part of the front panel of the garment and may be part of the back panel, too. The stretch banded area is adjacent the longitudinal ends of the front panel and the back panel to form a belt-like waistband. By removing the elastic strands from the longitudinal end area of the front panel, there is less retraction force - resulting in less force against the wearer’s skin and, therefore, a lower likelihood of red-marking of the skin.

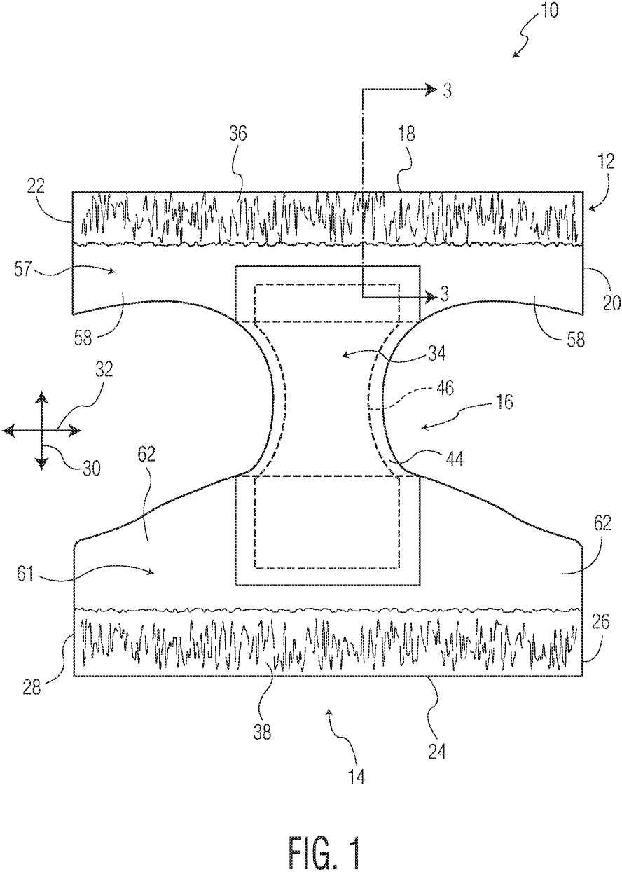

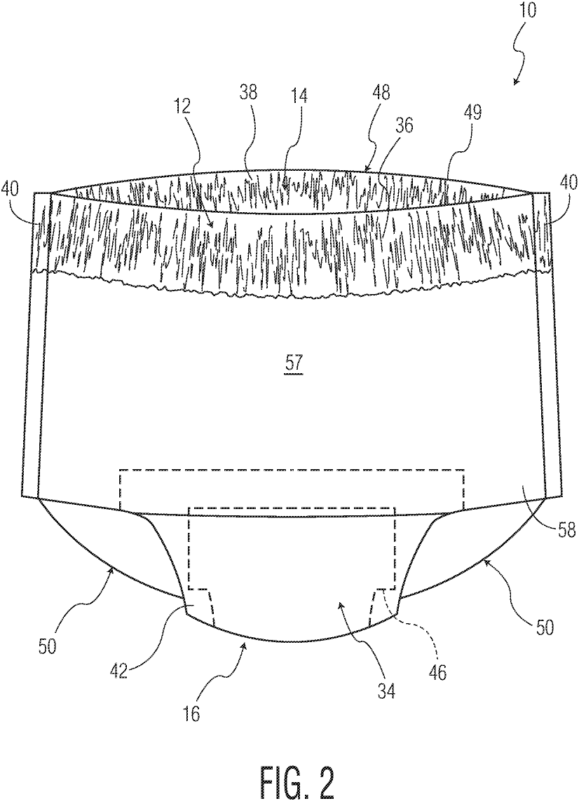

With reference to the drawings, FIGS. 1 and 2 illustrate one suitable aspect of an absorbent garment of the present disclosure in the form of a diaper pant, indicated generally at 10. While the present disclosure will be made in the context of the diaper pant 10, it should be understood that aspects of the present disclosure are applicable to other absorbent garments, such as, for example, toilet-training pants, youth pants, swim pants, feminine hygiene garments and adult incontinence garments. As used herein, the term “absorbent garment” refers to garments that absorb and contain body exudates and that are intended to be discarded after a limited period of use. The garments are not intended to be laundered or otherwise restored for reuse. Put differently, the garments are disposable. The garments can be placed against or in proximity to the body of a wearer to absorb and contain various exudates discharged from the body.

FIG. 1 illustrates the diaper pant 10 in an unfolded and laid flat condition to show an inner surface of the diaper pant that faces the wearer when the diaper pant 10 is worn. The diaper pant 10 has a longitudinal axis (direction) 30 and a lateral axis (direction) 32. Along the longitudinal axis 30, the diaper pant 10 defines a front region 12, a back region 14, and a crotch region 16 extending between and connecting the front region 12 and the back region 14.

In the front region 12, the diaper pant 10 has a front longitudinal end 18 and transversely opposed first and second front side edges 20, 22. A back longitudinal end 24 and transversely opposed first and second back side edges 26, 28 are located in the back region 14 of the diaper pant 10. In the illustrated aspect, the front longitudinal end 18 and the back longitudinal end 24 are straight edges. That is, the front longitudinal end 18 and the back longitudinal end 24 are substantially free from curves, bends, angles, notches or irregularities. It is understood, however, that the front longitudinal end 18 and/or the back longitudinal end 24 can be cut in any suitable shape as is known in the art (e.g., arcuate).

The diaper pant 10 includes a central absorbent assembly, indicated generally at 34, that extends longitudinally from the front region 12 through the crotch region 16 to the back region 14. The central absorbent assembly 34 of the illustrated aspect includes at least a bodyside liner 44 and an absorbent structure 46. In some embodiments, the central absorbent assembly 34 may further include an outer cover 42. In other embodiments, the outer cover 42 may be formed separately from the absorbent assembly 34 as part of the front and back panels 57, 61 and as part of a crotch panel (for example where the diaper pant 10 is formed as part of a “1-piece” construction, as is known in the art). As mentioned, for example where the diaper pant 10 is formed as part of a “3-piece” construction, as is known in the art, the absorbent assembly may comprise all of the bodyside liner 44, the outer cover 42, and the absorbent structure 46. In such embodiments, the bodyside liner 44 may be connected to the outer cover 42 in a superposed relation by suitable means such as adhesives, ultrasonic bonds, thermal bonds, pressure bonds, or combinations thereof with the absorbent structure 46 disposed between the liner 44 and the outer cover 44.

The front and back regions 12, 14 of the diaper pant 10 include a front panel 57 and a back panel, 61, respectively. The front panel 57 and the back panel 61 are interconnected via the absorbent assembly 34. The front panel 57 is a laminate or composite of several materials including an inner layer and an outer layer; the inner layer is closest to the skin of the wearer of the diaper pant 10. The front panel 57 has a front longitudinal end 18. Similarly, the back panel 61 is also a laminate or composite of several materials including an inner layer and an outer layer. The back panel 61 has a back longitudinal end 24. Between the inner layer and outer layer of the front panel 57, there is a stranded elastic area, described generally as elastic layer 70 or stranded elastic area 70 herein, and a stretch banded area 36. Between the inner layer and the outer layer of the back panel 61, there is a stranded elastic area 70. The stretch banded area 36 of the front panel 57 is disposed adjacent the front longitudinal end 18. The stranded elastic area 70 in the front panel 57 is adjacent to, but discrete from the stretch banded area 36 and the stranded elastic area 70 is closer to the crotch region 16 than the stretch banded area 36. The stretch banded area 36 has the appearance of a waistband, such as would be found on cloth underwear. As shown in FIG. 1, the back panel 61 may also have a stretch banded area 38 between the inner layer and the outer layer; the stretch banded area 38 of the back panel 61 is disposed adjacent the back longitudinal end 24.

By providing stretchability to the absorbent garment through the discrete areas of the stretch banded area 36 and the stranded elastic area 70, the absorbent garments of the present disclosure are more underwear-like in appearance because they have a noticeable waistband area, like underwear. The absorbent garments of the present disclosure also provide a more comfortable fit for the wearer because the stretch banded area 36 forming the waistband is softer and better-conforming.

As shown in FIG. 1, the front panel 57 includes a pair of laterally-opposed, front side portions 58 extending outward from the lateral center of the absorbent garment. Likewise, the back panel 61 incudes a pair of laterally-opposed, back side portions 62 extending outward from the lateral center of the absorbent garment. As shown in FIG. 2, the front panel 57 is joined to the back panel 61 by a pair of non-refastenable butt (or fin) seams 40 to define a pull-on, pant-like configuration of the diaper pant 10 having a waist opening, indicated at 48, and two leg openings, indicated at 50. More specifically, each front side portion 58 is joined to a respective back side portion 62 via one of the non-refastenable butt seams 40.

With the diaper pant 10 in the pull-on, pant-like configuration, illustrated in FIG. 2, the front region 12 includes the portion of the diaper pant 10 that, when worn, is positioned at least in part on the front of the wearer while the back region 14 includes the portion of the diaper pant 10 that is positioned at least in part on the back of the wearer. The crotch region 16 of the diaper pant 10 includes the portion of the diaper pant 10 that is positioned between the legs of the wearer and covers the lower torso of the wearer. The front and back side portions 58, 62 include the portions of the diaper pant 10 (and more particularly of the front and back regions 12, 14) that, when worn, are positioned on the hips of the wearer. As shown in FIG. 2, the stretch banded area 36 of the front panel 57 and the stretch banded area 38 of the back panel 61 cooperatively define the waist opening 48 of the diaper pant 10 that is configured to fully encircle the waist of the wearer.

The central absorbent assembly 34 is configured to contain and/or absorb exudates discharged from the wearer. The outer cover 42 suitably includes a material which is substantially liquid impermeable. The outer cover 42 can be a single layer of liquid impermeable material, but more suitably includes a multi-layered laminate structure in which at least one of the layers is liquid impermeable. For instance, the outer cover 42 can include a liquid permeable outer layer and a liquid impermeable inner layer that are suitably joined together by an adhesive, ultrasonic bonding, thermal bonding, pressure bonding, or combinations thereof. Suitable adhesives can be applied continuously or intermittently as beads, a spray, parallel swirls, or the like. The liquid permeable outer layer can be any suitable material, including materials that provide a generally cloth-like texture. The outer layer can also be made of those materials of which the liquid permeable bodyside liner 44 is made. While it is not a necessity for the outer layer to be liquid permeable, it is suitable that it provides a relatively cloth-like texture to the wearer.

The inner layer of the outer cover 42 can be both liquid and vapor impermeable, or it can be liquid impermeable and vapor permeable. The inner layer can be manufactured from a thin plastic film, although other flexible liquid impermeable materials can also be used. The inner layer, or the liquid impermeable outer cover 42 when a single layer, prevents waste material from wetting articles, such as bed sheets and clothing, as well as the wearer and caregiver.

If the outer cover 42 is a single layer of material, it can be embossed and/or matte finished to provide a more cloth-like appearance. As earlier mentioned, the liquid impermeable material can permit vapors to escape from the interior of the absorbent garment, while still preventing liquids from passing through the outer cover 42. One suitable “breathable” material is composed of a microporous polymer film or a nonwoven fabric that has been coated or otherwise treated to impart a desired level of liquid impermeability. It is also contemplated that the outer cover 42 can be stretchable, and more suitably elastic. In particular, the outer cover 42 is suitably stretchable and more suitably elastic in at least the transverse (lateral), or circumferential direction of the pant 10. In other aspects the outer cover 42 can be stretchable, and more suitably elastic, in both the transverse (lateral) and the longitudinal direction.

The liquid permeable bodyside liner 44 is illustrated as overlying the outer cover 42 and absorbent structure 46, and can, but need not, have the same dimensions as the outer cover 42. The bodyside liner 44 is suitably compliant, soft feeling, and non-irritating to the wearer’s skin. The bodyside liner 44 is also sufficiently liquid permeable to permit liquid body exudates to readily penetrate through its thickness to the absorbent structure 46. Further, the bodyside liner 44 can be less hydrophilic than the absorbent structure 46 to present a relatively dry surface to the wearer and permit liquid to readily penetrate through its thickness. The hydrophilic/hydrophobic properties can be varied across the length, width and/or depth of the bodyside liner 44 and absorbent structure 46 to achieve the desired wetness sensation or leakage performance.

The bodyside liner 44 can be manufactured from a wide selection of web materials, such as porous foams, reticulated foams, apertured plastic films, woven and nonwoven webs, or a combination of any such materials. For example, the bodyside liner 44 can include a meltblown web, a spunbonded web, or a bonded-carded-web composed of natural fibers, synthetic fibers or combinations thereof. The bodyside liner 44 can be composed of a substantially hydrophobic material, and the hydrophobic material can optionally be treated with a surfactant or otherwise processed to impart a desired level of wettability and hydrophilicity. The surfactant can be applied by any conventional means, such as spraying, printing, brush coating or the like. The surfactant can be applied to the entire bodyside liner 44 or can be selectively applied to particular sections of the bodyside liner 44, such as the medial section along the longitudinal center line.

The bodyside liner 44 can also be stretchable, and, more suitably, it can be elastomeric. In particular, the bodyside liner 44 is suitably stretchable and more suitably elastomeric in at least the lateral or circumferential direction 32 of the diaper pant 10. In other aspects, the bodyside liner 44 can be stretchable, and more suitably elastomeric, in both the lateral direction 32 and the longitudinal direction 30.

The absorbent structure 46 is suitably compressible, conformable, non-irritating to a wearer’s skin, and capable of absorbing and retaining liquids and certain body wastes. For example, the absorbent structure 46 can include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof.

The materials can be formed into a web structure by employing various conventional methods and techniques. For example, the absorbent structure 46 can be formed by a dry-forming technique, an air forming technique, a wet-forming technique, a foam-forming technique, or the like, as well as combinations thereof. Furthermore, the absorbent structure 46 can itself encompass multiple layers in a vertical direction 33 (e.g., thickness) of the absorbent structure 46. Such multiple layers can take advantage of differences in absorbent capacity, such as by placing a lower absorbent capacity material layer closer to the bodyside liner 44 and a higher absorbent capacity material closer to the outer cover 42. Likewise, discrete portions of a single-layered absorbent structure can encompass higher capacity absorbents, and other discrete portions of the structure can encompass lower capacity absorbents.

Superabsorbent material is suitably present in the absorbent structure 46 in an amount of from about 0 to about 100 weight percent based on total weight of the absorbent structure 46. The absorbent structure 46 can suitably have a density within the range of about 0.10 to about 0.60 grams per cubic centimeter. Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and preferably is capable of absorbing more than about 25 times its weight in liquid.

The absorbent structure 46 can alternatively include a coform material. The term “coform material” generally refers to composite materials including a mixture or stabilized matrix of thermoplastic fibers and a second non-thermoplastic material. As an example, coform materials are made by a process in which at least one meltblown die head is arranged near a chute through which other materials are added to the web while it is forming. Such other materials can include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers can be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene.

In one suitable aspect, the absorbent structure 46 is stretchable so as not to inhibit the stretchability of other components to which the absorbent structure can be adhered, such as the outer cover 42 and the bodyside liner 44. After being formed or cut to a desired shape, the absorbent structure 46 can be wrapped or encompassed by a suitable wrap (not shown) that aids in maintaining the integrity and shape of the absorbent structure 46.

The absorbent assembly 34 can also include a surge management layer (not shown) located adjacent the absorbent structure 46 (e.g., between the absorbent structure 46 and the bodyside liner 44) to help decelerate and diffuse surges or gushes of liquid that can be rapidly introduced into the absorbent structure 46 of the diaper pant 10 by the wearer. Desirably, the surge management layer can rapidly accept and temporarily hold the liquid prior to releasing the liquid into the storage or retention portions of the absorbent structure 46. Examples of suitable surge management layers are described in U.S. Pat. No. 5,486,166 issued Jan. 23, 1996 to Bishop et al.; U.S. Pat. No. 5,490,846 issued Feb. 13, 1996 to Ellis et al.; and U.S. Pat. No. 5,820,973 issued Oct. 13, 1998 to Dodge, II et al., the entire disclosures of which are hereby incorporated by reference.

The absorbent assembly 34 can also include a pair of containment flaps (not shown) that extend longitudinally along the absorbent assembly 34 and are adapted to provide a barrier to the lateral flow of body exudates as is known in the art. The containment flaps can be connected to the bodyside liner 44 or other components of the absorbent assembly 34. Suitable configurations of the containment flaps are described, for example, in U.S. Pat. No. 5,599,338 issued Feb. 4, 1997, to K. Enloe, the entirety of which is incorporated herein by reference.

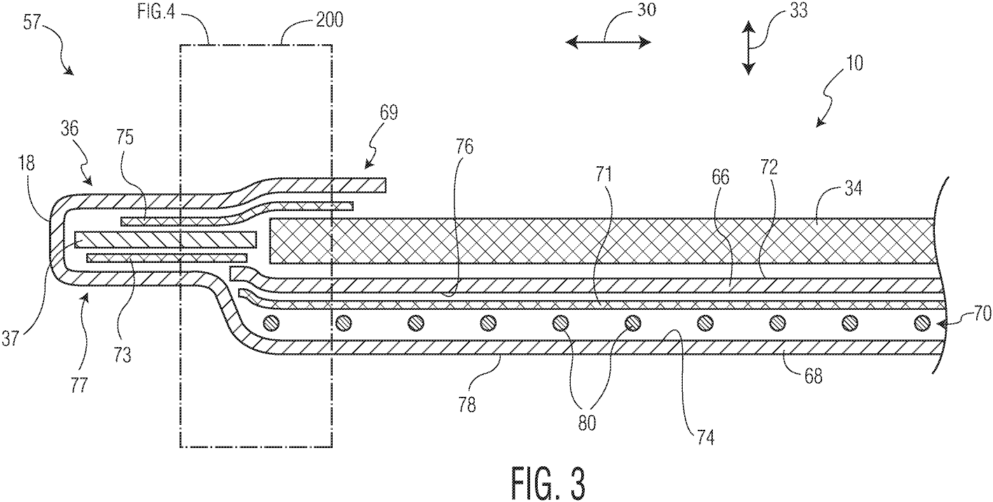

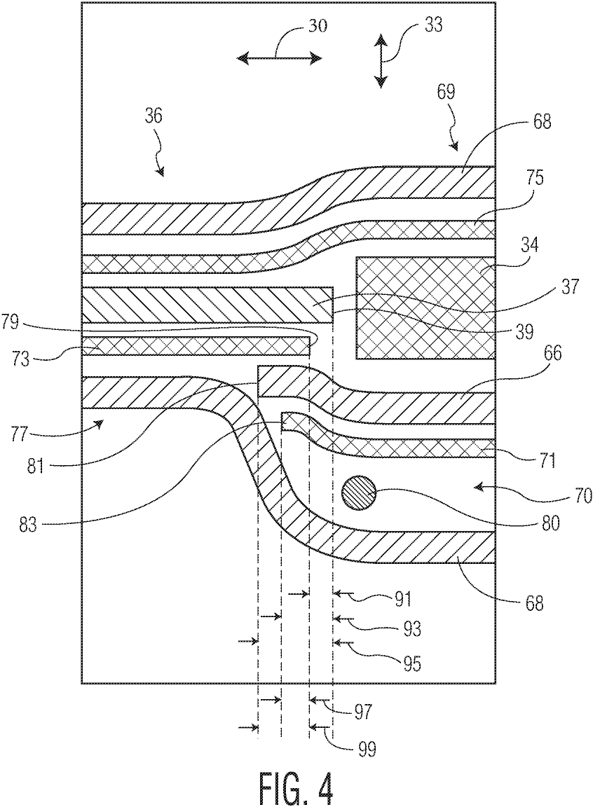

With reference to FIGS. 3 and 4, the structure used to form the front panel 57 includes a multi-layer construction including an inner or body-facing layer 66, an outer or garment-facing layer 68, and an elastic layer 70 disposed between the inner layer 66 and the outer layer 68. Although not shown in FIGS. 3 and 4, the construction of the back panel 61 may be substantially similar to that described with respect to the front panel 57 in FIGS. 3 and 4.

The inner layer 66 and the outer layer 68 each include, respectively, a body-facing side 72, 74 and a garment-facing side 76, 78. The garment-facing side 76 of the inner layer 66 is connected to the body-facing side 74 of the outer layer 68 by suitable means such as adhesives, ultrasonic bonds, thermal bonds, pressure bonds, or combinations thereof. In the illustrated aspect, an upper portion 69 of the outer layer 68 is folded over an end 81 of the inner layer 66. The upper portion 69 can be connected to the body-facing side 72 of the inner layer 66 and may at least partially enclose the elastic layer 70 between the inner layer 66 and the outer layer 68.

The inner layer 66 is typically constructed from a non-apertured nonwoven material. Suitable nonwovens include single layer nonwovens, such as spunbond webs, and nonwoven laminates. In one suitable aspect, the inner layer 66 includes a multiple layer spunbond laminate material. As used herein, the term SSS may refer a 3-layer spunbond laminate (e.g. a spunbond/spunbond/spunbond laminate), while the term SSSS may refer a 4-layer spunbond laminate (e.g. a spunbond/spunbond/spunbond/spunbond laminate). In another suitable aspect, the inner layer 66 includes at least one meltblown layer positioned between two or more spunbond layers to form a spunbond/meltblown/spunbond (“SMS”) laminate. In one particular aspect, the inner layer 66 includes a spunbond/spunbond/meltblown/meltblown/spunbond laminate (“SSMMS”). The nonwoven laminate can have other configurations and possess any desired number of meltblown and spunbond layers, such as spunbond/meltblown/meltblown/spunbond laminates (“SMMS”), spunbond/meltblown laminates (“SM”), etc. In addition to or as an alternative to meltblown and spunbond webs, a variety of other nonwoven webs can also be used to form the inner layer 66 including, for example and without limitation, through-air bonded carded webs (TABCW), thermally bonded carded webs, wet-laid webs, coform webs, and hydraulically entangled webs. Some suitable exemplary materials for inner layer 66 include a TABCW material having a basis weight of between about 15 gsm and about 25 gsm, an SMS material having a basis weight of between about 10 gsm and about 20 gsm, and a SSSS material having a basis weight of between about 10 gsm and about 20 gsm.

The inner layer 66 may be liquid-impermeable and vapor permeable. That is, the inner layer 66 permits vapors to escape from the interior of the absorbent garment, while still preventing liquids from passing through the inner layer 66. The inner layer 66 can be treated or otherwise processed to impart a desired level of liquid impermeability to the inner layer 66.

The elastic layer 70 is attached to at least one of the inner layer 66 and the outer layer 68 to impart a desired level of elasticity to the front panel 57. The elastic layer 70 may be attached to the inner layer 66 and/or the outer layer 68 by any suitable means including, for example, adhesives such as adhesive 71. The elastic layer 70 may be stretched and then adhered to one or both of the inner layer 66 and the outer layer 68, or adhered to one or both of the inner layer 66 and the outer layer 68 when the layers 66, 68 are in a gathered state to impart a desired level of elasticity to the front panel 57. In other aspects, the elastic layer 70 may be adhered to one or both of the inner layer 66 and the outer layer 68, and then elasticized or shrunk, for example with the application of heat, such that elastic retractive forces are imparted to the front panel 57.

The elastic layer 70 may be formed of a variety of suitable elastic materials, including strands or ribbons of natural rubber, synthetic rubber, or thermoplastic elastomeric polymers. In the aspect shown in FIGS. 3 and 4, the elastic layer 70 includes a plurality of elastomeric strands 80 extending in the lateral direction 32 (FIG. 1) or circumferential direction (FIG. 2) of the diaper pant 10. In one suitable aspect, the elastomeric strands 80 include dry-spun coalesced multifilament spandex elastomeric strands sold under the trade name LYCRA® and available from Invista of Wichita, Kansas, U.S.A. Other suitable materials from which the elastic layer 70 may be constructed include vertical filament laminate (VFL) materials, an example of which is described in U.S. Pat. No. 6,916,750 to Thomas et al., which is hereby incorporated by reference.

The outer layer 68 is vapor permeable, and may be liquid permeable or liquid impermeable. The outer layer 68 may be constructed from an apertured nonwoven, such as a single layer nonwoven or a nonwoven laminate. In one suitable aspect, the outer layer 68 includes a spunbond/spunbond/spunbond (“SSS”) laminate. In another suitable aspect, the outer layer 68 includes at least one meltblown layer positioned between two or more spunbond layers to form a spunbond/meltblown/spunbond (“SMS”) laminate. In one aspect, the outer layer 68 includes a spunbond/spunbond/meltblown/meltblown/spunbond laminate (“SSMMS”). The nonwoven laminate can have other configurations and possess any desired number of meltblown and spunbond layers, such as spunbond/meltblown/meltblown/spunbond laminates (“SMMS”), spunbond/meltblown laminates (“SM”), etc. In addition to or as an alternative to meltblown and spunbond webs, a variety of other nonwoven webs can also be used to form the outer layer 68 including, for example and without limitation, through-air bonded carded webs, thermally bonded carded webs, wet-laid webs, coform webs, and hydraulically entangled webs.

In the example of FIGS. 3 and 4, the elastic layer 70 is attached to the inner and outer layers 66, 68 by adhesive 71, sometimes termed a layer construction adhesive or an inner layer adhesive herein. Adhesive 71 further operates to couple the inner layer 66 and the outer layer 68 together in a face-to-face relationship. Adhesive 71 may be applied continuously or intermittently as beads, a spray, parallel swirls, or the like. Additionally, although shown adjacent the inner layer 66, the adhesive 71 may be applied to either the inner layer 66, the outer layer 68, or to both layers 66, 68. The adhesive 71 may be any suitable construction adhesive and may operate to adhere all of the layers 66, 68 and the elastic layer 70 together. In some embodiments, the adhesive 71 may even be an elastomeric adhesive (i.e. materials capable of at least 75% elongation without rupture), such as aqueous-based styrene butadiene adhesives, neoprene, polyvinyl chloride, vinyl copolymers, polyamides, and ethylene vinyl terpolymers.

In other embodiments, however, the inner layer 66 and the outer layer 68 may be joined together by suitable means other than by adhesives, such as through ultrasonic bonding, thermal bonding, pressure bonding, or combinations thereof. In such embodiments, or even in embodiments where adhesive 71 operates to join the layers 66, 68 together, the elastic strands 80 of the elastic layer 70 may have additional adhesive (not shown) applied directly to the elastic members - for example a strand adhesive may be applied to individual strands 80 of the elastic layer 70.

In some embodiments according to aspects of the present disclosure, such as is shown in FIGS. 3 and 4, pant 10 may further comprise a stretch banded area 36 formed of stretch material 37 disposed adjacent the stranded elastic layer 70. As shown, the stretch banded area 36 can be separate, in the longitudinal direction 30, from the stranded elastic layer 70. That is, the stretch banded area 36 can be spaced apart in the longitudinal direction 30 from the elastomeric strands 80 of the elastic layer 70. Although, in other embodiments, one or two of the elastomeric strands 80 may overlap the stretch banded area 36 in the vertical direction 33.

The stretch material 37, sometimes termed stretch band material herein, forming part of the stretch banded area 36 may be formed of films, film laminates and apertured elastic films, examples of which are described in U.S. Pat. No. 7,803,244 issued Sep. 28, 2010 to Siqueira et al., and U.S. Pat. No. 8,361,913 issued Jan. 29, 2013 to Siqueira et al., both of which are hereby incorporated by reference, and other elastic laminates such as single- and dual-faced spandex laminates, stretch-bonded laminates (SBL), and continuous filament stretch-bonded laminates (CFSBL), simultaneously aperture and bonded bi-directional elastic laminate (SABBEL), examples of which are described in U.S. Pat. No. 5,385,775 issued Jan. 31, 1995 to Wright; U.S. Pat. No. 6,057,024 issued May 2, 2000 to Mleziva et al.; and U.S. Pat. No. 6,969,441 issued Nov. 29, 2005 to Welch et al., all of which are hereby incorporated by reference. The elastic material 37 can be stretched and bonded to a substrate, bonded to a gathered substrate, or bonded to a substrate and then elasticized or shrunk, for example, with the application of heat, such that elastic retractive forces are imparted to the substrate. In general, the elastic material 37 may be form a ribbon or band having a non-round cross-section.

As shown in FIGS. 3 and 4, the outer layer 68 may have an upper portion 69 that is folded over a portion of the stretch banded area 36. The upper portion 69 may further be folded over a portion of the elastic layer 70. That is, the upper portion 69 may overlap, in the vertical direction 33, a portion of the elastic layer 70. The upper portion 69 may further overlap a portion of the absorbent assembly 34 in some embodiments.

The upper portion 69 may be secured to the stretch material 37 by adhesive 75. In some embodiments, the adhesive 75 may additionally attached the upper portion 69 of the outer layer 68 to a portion of the absorbent assembly 34. Although, this is not required in all cases. Depending on the longitudinal spacing between the stretch banded area 36 and the elastic layer 70, the adhesive 75 may further operate to couple a part of the upper portion 69 to the inner layer 66 between the stretch banded area 36 and the elastic layer 70. The adhesive 75 may generally be any suitable construction adhesive and may be present in an amount between about 0.25 gsm and about 5 gsm.

The stretch material 37 may be coupled to a bottom portion 77 of the outer layer 68 by adhesive 73, sometimes termed stretch material adhesive herein. In contrast to adhesive 75, the adhesive 73 may be any suitable elastic attachment adhesive. In at least some embodiments, the adhesive 75 may be applied as lines of adhesive, or swirls comprising individual adhesive fibers. Where the adhesive 75 is applied in such a manner, the adhesive 75 may be present in an amount between about 1.0 g/m and about 3.0 g/m, or between about 1.5 g/m and about 2.5 g/m. In some embodiments, as best seen in FIG. 4, the adhesive 73 may have an end 79 disposed proximate the absorbent assembly 34 and which has an alignment with the end 39 of the stretch material 37. A longitudinal separation between the end 79 of the adhesive 73 and the end 39 of the stretch material 37 is shown in FIG. 4 as distance 91. According to some embodiments of the present disclosure, the distance 91 may be between about 0 mm and about 10 mm, or between about 2 mm and about 10 mm, or between about 2 mm and about 8 mm, or between about 2 mm and about 6 mm.

In order to maintain a secure connection between all of the layers of the front panel 57, and especially between the upper portion 69 of the outer layer 68, the stretch material 37, and the inner layer 66, the various layers and materials 66, 68, 37 and adhesives 71, 73 should have sufficient overlaps, as described in more detail below. Failing to provide for such described overlaps between the various layers and materials 66, 68, 37 and adhesives 71, 73 can result in delamination of the various layers and materials 66, 68, 37 due to forces imparted onto the pant 10 during manufacturing and/or wear. These overlap distances are particularly suited where the stretch material 37 has an overall longitudinal length of between about 20 mm and about 40 mm.

Accordingly, as best seen in FIG. 4, overlap distance 93 defines the longitudinal overlap between the end 39 of the stretch material 37 and the end 83 of the adhesive 71. The end 83 of the adhesive 71 is the portion of adhesive 71 disposed most closely to the front longitudinal end 18 of the pant 10. Overlap distance 95 defines the longitudinal overlap between the end 39 of the stretch material 37 and the end 81 of the inner layer 66. Overlap distance 97 defines the longitudinal overlap between the end 79 of the adhesive 73 and the end 83 of the adhesive 71. Finally, overlap distance 99 defines the longitudinal overlap between the end 79 of the adhesive 73 and the end 81 of the inner layer 66.

According to one aspect of the present disclosure, it is preferable that the overlap distance 93 be greater than about 40% of the overall length of the stretch material 37 in the longitudinal direction 30. In further embodiments, it may be preferable that the overlap distance 93 be greater than about 45%, or greater than about 50%, or greater than about 55%, or greater than about 60% of the overall length of the stretch material 37. It has been found that an overlap distance of less than about 40% can result in undesirable delamination of the layers 66, 68 and/or the stretch material 37. Additionally, it may be preferable that the overlap distance 93 to be less than about 80%, or less than about 70%, or less than about 60% of the overall length of the stretch material 37. For example, it may preferable for the overlap distance 93 to be greater than about 40% and less than about 80% of the overall length of the stretch material 37, or greater than about 50% and less than about 60% of the overall length of the stretch material 37, or any other combination of the above described ranges.

It has further been found that different overlap distances 93 may be suitable for differing materials. For example, a stronger material (for example as measured by a CD tensile strength measurement of a material) may allow for relatively lower overlap distances 93 than weaker materials to still achieve a desired lamination strength between the layers 66, 68 and the stretch material 37. As one example, it has been found that products utilizing TABCW materials in the 15 gsm to 25 gsm basis weight ranges having an overlap distance of less than about 40% did not have sufficient lamination strengths. In fact, there did not appear to be any appreciable difference in lamination strengths between embodiments where the overlap distance 93 was about 17% and where the overlap distance was about 42% of the overall length of the stretch material 37, in such examples. Accordingly, in embodiments where the inner layer 66 is a TABCW material having a basis weight of between about 15 gsm and about to 25 gsm, the overlap distance 93 may preferably be greater than about 60%, or greater than about 70%, or greater than about 80% of the overall length of the stretch material 37. Additionally, when using TABCW materials having a basis weight of between about 10 gsm and about 30 gsm, it may be preferable that the overlap distance 93 to be less than about 90% or less than about 80% of the overall length of the stretch material 37. For example, it may preferable for the overlap distance 93 to be greater than about 60% and less than about 90% of the overall length of the stretch material 37, or greater than about 70% and less than about 80% of the overall length of the stretch material 37, or any other combination of the above described ranges.

Conversely, stronger materials may require lesser overlap distance 93. For example, it has been found that SSSS materials in the 10 gsm to 20 gsm basis weight range had sufficient lamination strength where the overlap distance 93 was greater than about 40% of the overall length of the stretch material 37. Still, in such embodiments it may be preferable for the overlap distance 93 to be greater than about 45%, or greater than about 50%, or greater than about 55%, or greater than about 60% of the overall length of the stretch material 37. Additionally, when using SSSS material having a basis weight of between about 10 gsm and about 20 gsm, it may be preferable that the overlap distance 93 to be less than about 80%, or less than about 70%, or less than about 60% of the overall length of the stretch material 37. For example, it may preferable for the overlap distance 93 to be greater than about 40% and less than about 80% of the overall length of the stretch material 37, or greater than about 50% and less than about 60% of the overall length of the stretch material 37, or any other combination of the above described ranges.

Expanding on the above described examples more broadly, it has been found that materials used for inner layer 66 having a CD tensile strength value of greater than about 12N/50mm, along with the overlap distance 93 being greater than about 40% of the overall length of the stretch material 37 will produce a pant 10 having a sufficient lamination strength, regardless of the specific material used. Although, in some embodiments, it may be desirable to use an overlap distance 93 of greater than about 45%, or greater than about 50%, or greater than about 55%, or greater than about 60% of the overall length of the stretch material 37 in order to ensure a desired lamination strength. In such embodiments, the CD tensile strength value may usually be less than about 25 N/50 mm.

Where materials used for inner layer 66 have lower CD tensile strengths, less than about 8.0 N/50 mm, the overlap distance 93 being may need to be greater than about 60% of the overall length of the stretch material 37 to provide sufficient lamination strength. Although, in some embodiments, it may be desirable to use an overlap distance 93 greater than about 70%, or greater than about 80% of the overall length of the stretch material 37 in order to ensure a desired lamination strength. Also in such embodiments, the CD tensile strength value may usually be less than about 25 N/50 mm.

As described, the overlap distance 97 describes the overlap distance between the end 79 of the adhesive 73 and the end 83 of the adhesive 71. Where the ends 81 (of the inner layer 66) and 83 align in the vertical direction 33 and the ends 79 (of the adhesive 73) and the end 39 (of the stretch material 37) align in the vertical direction, the overlap distance 97 may be the same as any of the above described overlap distances 93. Accordingly, in such embodiments, the overlap distance 97 may have the same preferred ranges as described above with respect to overlap distance 93. However, where the ends 81 and 83 do not align and/or the ends 79 and 39 do not align, it still may be preferable for the overlap distance 97 to have any of the ranges described above with respect to overlap distance 93. Where the ends 81 and 83 do not align and/or the ends 79 and 39 do not align, the difference in positioning between the ends 81 and 83 or the ends 79 and 39 may not generally exceed about 15 mm.

The overlap distance 95, as detailed above, describes the overlap distance between the end 39 of the stretch material 37 and the end 81 of the inner layer 66. Where the ends 81 and 83 align in the vertical direction 33, the overlap distance 95 may be the same as any of the above described overlap distances 93. Accordingly, in such embodiments, the overlap distance 97 may have the same preferred ranges as described above with respect to overlap distance 93. Where the ends 81 and 83 do not align, the overlap distance 95 may generally be greater than the overlap distance 93. In at least some embodiments, it may be preferable for the overlap distance 95 to not exceed the overlap distance 93 by more than about 50%, or more than about 25% in other embodiments.

The overlap distance 99, as detailed above, describes the overlap distance between the end 79 of the adhesive 73 and the end 81 of the inner layer 66. Where the ends 79 and 39 align in the vertical direction 33, the overlap distance 99 may be the same as any of the above described overlap distances 95. Accordingly, in such embodiments, the overlap distance 99 may have the same preferred ranges as described above with respect to overlap distance 95. Where the ends 79 and 39 do not align, the overlap distance 99 may generally be less than the overlap distance 93. Where the overlap distance 99 is less than the overlap distance 95, it may be preferable for the difference between the overlap distance 99 and the overlap distance 95 to not exceed about 50%, or to not exceed about 25% in other embodiments.

In the interests of brevity and conciseness, any ranges of values set forth in this disclosure contemplate all values within the range and are to be construed as support for claims reciting any sub-ranges having endpoints that are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by references, the meaning or definition assigned to the term in this written document shall govern.

While particular aspects of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

### Embodiments

According to a first embodiment, an absorbent garment may comprise a longitudinal axis and a lateral axis, a front region, a back region, a crotch region, the crotch region being disposed between the front region and the back region, and the crotch region including an absorbent assembly, a front panel in the front region, wherein the front panel includes an inner layer, an outer layer and a front longitudinal end, a back panel in the back region, wherein the back panel includes an inner layer, an outer layer and a back longitudinal end, a stranded elastic area disposed between the inner layer and the outer layer in the front panel and in the back panel, a stretch banded area comprising a stretch band material disposed between the outer layer and at least a portion of the inner layer in the front panel, the stretch band material disposed adjacent the longitudinal end of the front panel with the stranded elastic area adjacent and discrete from the stretch band material and disposed closer to the crotch region than the stretch band material, and a stretch band material adhesive coupling the stretch band material to the front panel inner layer, and wherein a longitudinal overlap between the stretch band material adhesive and the front panel inner layer may be greater than about 40% of an overall longitudinal length of the stretch band material.

According to a second embodiment, the longitudinal overlap between the stretch band material adhesive and the front panel inner layer of the first embodiment may be greater than about 50% of the overall longitudinal length of the stretch band material.

According to a third embodiment, the longitudinal overlap between the stretch band material adhesive and the front panel inner layer of the first embodiment may be greater than about 60% of the overall longitudinal length of the stretch band material.

According to a fourth embodiment, the longitudinal overlap between the stretch band material adhesive and the front panel inner layer of any of the first through third embodiments may be less than about 80% of the overall longitudinal length of the stretch band material.

According to a fifth embodiment, the article of any of the first through fourth embodiments may further comprise a front panel inner layer adhesive coupling the front panel inner layer to the front panel outer layer, and a longitudinal overlap between the front panel inner layer adhesive and the stretch band material may be greater than about 40% of the overall longitudinal length of the stretch band material.

According to a sixth embodiment, the longitudinal overlap between the inner layer adhesive and the stretch band material of the fifth embodiment may be greater than about 50% of the overall longitudinal length of the stretch band material.

According to a seventh embodiment, the longitudinal overlap between the inner layer adhesive and the stretch band material of any of the fifth or sixth embodiments may be less than about 80% of the overall longitudinal length of the stretch band material.

According to an eighth embodiment, the stretch band material adhesive of any of the fifth through seventh embodiments may be an elastic attachment adhesive and the inner layer adhesive may be a construction adhesive.

According to a ninth embodiment, the front panel inner layer of any of the first through eight embodiments may comprise a material having a CD tensile strength of greater than about 12 N/50 mm.

According to a tenth embodiment, the front panel inner layer of the third embodiment may comprise a material having a CD tensile strength of greater than about 8.0 N/50 mm.

According to an eleventh embodiment, the front panel inner layer of the ninth embodiment may comprise a multi-layer spunbond material having a basis weight of between about 10 gsm and about 30 gsm.

According to a twelfth embodiment, the front panel inner layer of the tenth embodiment may comprise front panel inner layer comprises a TABCW material having a basis weight of between about 15 gsm and about 25 gsm.

According to a thirteenth embodiment, the article of any of the first through twelfth embodiments may further including a stretch banded area comprising a stretch band material disposed between the inner layer and the outer layer in the back panel, wherein the stretch band material is disposed adjacent the longitudinal end of the back panel.

In a fourteenth embodiment, an absorbent garment may comprise a longitudinal axis and a lateral axis, a front region, a back region, a crotch region, the crotch region being disposed between the front region and the back region, and the crotch region including an absorbent assembly, a front panel in the front region, wherein the front panel includes an inner layer, an outer layer and a front longitudinal end, the front panel inner layer comprising a material having a CD tensile strength that is greater than about 12 N/50 mm, a back panel in the back region, wherein the back panel includes an inner layer, an outer layer and a back longitudinal end, a stranded elastic area disposed between the inner layer and the outer layer in the front panel and in the back panel, a stretch banded area comprising a stretch band material disposed between the outer layer and at least a portion of the inner layer in the front panel, the stretch band material disposed adjacent the longitudinal end of the front panel with the stranded elastic area adjacent and discrete from the stretch band material and disposed closer to the crotch region than the stretch band material, an inner layer adhesive coupling the front panel inner layer to the front panel outer layer, and a stretch band material adhesive coupling the stretch band material to the front panel inner layer, and wherein a longitudinal overlap between the inner layer adhesive and the stretch band material adhesive may be greater than about 40% of an overall longitudinal length of the stretch band material.

According to a fifteenth embodiment, the longitudinal overlap between the inner layer adhesive and the stretch band material adhesive of the fourteenth embodiment may be greater than about 50% of the overall longitudinal length of the stretch band material.

According to a sixteenth embodiment, the longitudinal overlap between the inner layer adhesive and the stretch band material adhesive of the fourteenth embodiment may be greater than about 60% of the overall longitudinal length of the stretch band material.

According to a seventeenth embodiment, the front panel inner layer of the sixteenth embodiment may comprise a material having a CD tensile strength that is greater than about 8.0 N/50 mm.

According to an eighteenth embodiment, an end of the stretch band material adhesive disposed proximate the absorbent assembly and an end of the stretch band material disposed proximate the absorbent assembly of any of the fourteenth through seventeenth embodiments may be un-aligned in a vertical direction that is perpendicular to the lateral axis and the longitudinal axis.

According to a nineteenth embodiment, an end of the front panel inner layer disposed proximate the front longitudinal end and an end of the inner layer adhesive disposed proximate front longitudinal end of any of the fourteenth through eighteenth embodiments may be un-aligned in a vertical direction that is perpendicular to the lateral axis and the longitudinal axis.

According to a twentieth embodiment, in any of the fourteenth through nineteenth embodiments, the stretch band material adhesive may be an elastic attachment adhesive and the inner layer adhesive may be a construction adhesive.

We claim:

1. An absorbent garment comprising:
a longitudinal axis and a lateral axis;
a front region, a back region, a crotch region, the crotch region being disposed between the front region and the back region, and the crotch region including an absorbent assembly;
a front panel in the front region, wherein the front panel includes an inner layer, an outer layer and a front longitudinal end;
a back panel in the back region, wherein the back panel includes an inner layer, an outer layer and a back longitudinal end;
a stranded elastic area disposed between the inner layer and the outer layer in the front panel and in the back panel;
a stretch banded area comprising a stretch band material disposed between the outer layer and at least a portion of the inner layer in the front panel, the stretch band material disposed adjacent the longitudinal end of the front panel with the stranded elastic area adjacent and discrete from the stretch band material and disposed closer to the crotch region than the stretch band material; and
a stretch band material adhesive coupling the stretch band material to the front panel inner layer;
wherein a longitudinal overlap between the stretch band material adhesive and the front panel inner layer is greater than about 40% of an overall longitudinal length of the stretch band material.

2. The absorbent article of claim 1, wherein the longitudinal overlap between the stretch band material adhesive and the front panel inner layer is greater than about 50% of the overall longitudinal length of the stretch band material.

3. The absorbent article of claim 1, wherein the longitudinal overlap between the stretch band material adhesive and the front panel inner layer is greater than about 60% of an overall longitudinal length of the stretch band material.

4. The absorbent article of claim 2, wherein the longitudinal overlap between the stretch band material adhesive and the front panel inner layer is less than about 80% of the overall longitudinal length of the stretch band material.

5. The absorbent article of claim 1, further comprising a front panel inner layer adhesive coupling the front panel inner layer to the front panel outer layer, and wherein a longitudinal overlap between the front panel inner layer adhesive and the stretch band material is greater than about 40% of the overall longitudinal length of the stretch band material.

6. The absorbent article of claim 5, wherein the longitudinal overlap between the inner layer adhesive and the stretch band material is greater than about 50% of the overall longitudinal length of the stretch band material.

7. The absorbent article of claim 5, wherein the longitudinal overlap between the inner layer adhesive and the stretch band material is less than about 80% of the overall longitudinal length of the stretch band material.

8. The absorbent article of claim 5, wherein the stretch band material adhesive is an elastic attachment adhesive and wherein the inner layer adhesive is a construction adhesive.

9. The absorbent article of claim 1, wherein the front panel inner layer comprises a material having a CD tensile strength of greater than about 12 N/50 mm.

10. The absorbent article of claim 3, wherein the front panel inner layer comprises a material having a CD tensile strength of greater than about 8.0 N/50 mm.

11. The absorbent article of claim 9, wherein the front panel inner layer comprises a multi-layer spunbond material having a basis weight of between about 10 gsm and about 30 gsm.

12. The absorbent article of claim 10, wherein the front panel inner layer comprises a TABCW material having a basis weight of between about 15 gsm and about 25 gsm.

13. The absorbent article of claim 1, further including a stretch banded area comprising a stretch band material disposed between the inner layer and the outer layer in the back panel, wherein the stretch band material is disposed adjacent the longitudinal end of the back panel.

14. An absorbent garment comprising:
a longitudinal axis and a lateral axis;
a front region, a back region, a crotch region, the crotch region being disposed between the front region and the back region, and the crotch region including an absorbent assembly;
a front panel in the front region, wherein the front panel includes an inner layer, an outer layer and a front longitudinal end, the front panel inner layer comprising a material having a CD tensile strength that is greater than about 12 N/50 mm;
a back panel in the back region, wherein the back panel includes an inner layer, an outer layer and a back longitudinal end;
a stranded elastic area disposed between the inner layer and the outer layer in the front panel and in the back panel;
a stretch banded area comprising a stretch band material disposed between the outer layer and at least a portion of the inner layer in the front panel, the stretch band material disposed adjacent the longitudinal end of the front panel with the stranded elastic area adjacent and discrete from the stretch band material and disposed closer to the crotch region than the stretch band material;
an inner layer adhesive coupling the front panel inner layer to the front panel outer layer; and
a stretch band material adhesive coupling the stretch band material to the front panel inner layer;
wherein a longitudinal overlap between the inner layer adhesive and the stretch band material adhesive is greater than about 40% of an overall longitudinal length of the stretch band material.

15. The absorbent article of claim 14, wherein the longitudinal overlap between the inner layer adhesive and the stretch band material adhesive is greater than about 50% of the overall longitudinal length of the stretch band material.

16. The absorbent article of claim 14, wherein the longitudinal overlap between the inner layer adhesive and the stretch band material adhesive is greater than about 60% of the overall longitudinal length of the stretch band material.

17. The absorbent article of claim 16, wherein the front panel inner layer comprises a material having a CD tensile strength that is greater than about 8.0 N/50 mm.

18. The absorbent article of claim 14, wherein an end of the stretch band material adhesive disposed proximate the absorbent assembly and an end of the stretch band material disposed proximate the absorbent assembly are un-aligned in a vertical direction, the vertical direction being perpendicular to the lateral axis and the longitudinal axis.

19. The absorbent article of claim 14, wherein an end of the front panel inner layer disposed proximate the front longitudinal end and an end of the inner layer adhesive disposed proximate front longitudinal end are un-aligned in a vertical direction, the vertical direction being perpendicular to the lateral axis and the longitudinal axis.

20. The absorbent article of claim 14, wherein the stretch band material adhesive is an elastic attachment adhesive and wherein the inner layer adhesive is a construction adhesive.
